(19) United States
(12) Patent Application Publication
(10) Pub. No.: US 2026/0128141 A1
(43) Pub. Date: May 7, 2026

(54) INTELLIGENT HEALTH MANAGEMENT SYSTEMS AND METHODS WITH REAL-TIME DATA INTEGRATION AND PERSONALIZED REPORTING

(71) Applicants: Kian Hong QUAH, Pleasanton, CA (US); Wee Lip NG, Johor Bahru (MY)

(72) Inventors: Kian Hong QUAH, Pleasanton, CA (US); Wee Lip NG, Johor Bahru (MY)

(21) Appl. No.: 19/382,405

(22) Filed: Nov. 7, 2025

(30) Foreign Application Priority Data
Nov. 7, 2024 (MY) PI2024006397

(51) Int. Cl. G16H 15/00; G16H 50/30

(52) U.S. Cl. CPC G16H 15/00; G16H 50/30

(57) ABSTRACT

A method for intelligent health management with real-time data integration and personalized reporting includes the steps of: collecting user-specific data from a plurality of sources, wherein the user-specific data comprises biometric data, clinical data, psychological data, lifestyle data, and social support data; processing the collected data using a hybrid AI engine that integrates quantitative analytical methods with large language models (LLMs); generating a reconfigurable hierarchical decision matrix that dynamically incorporates both qualitative and quantitative decision-making processes; producing personalized health reports through an output module, wherein the reports comprise actionable insights, risk assessments, and individualized recommendations tailored to user preferences and medical history; and, automatically reconfiguring decision guidelines and thresholds in real-time based on, new medical research data; user-specific health variations; and, healthcare provider inputs and feedback.

### CROSS-REFERENCE TO RELATED APPLICATION

This application claims the benefit of and priority to Malaysian patent application no. PI2024006397 filed on 7 Nov. 2024, which are herein incorporated by reference in its entirety.

### BACKGROUND OF INVENTION

As it is well known, over the past decade, the integration of digital technologies in healthcare has led to substantial improvements in diagnostics, remote monitoring, and preventive care. The modern wearable devices now provide continuous monitoring capabilities, as these devices collect physiological data including heart rate, oxygen saturation, sleep patterns, and physical activity levels, enabling real-time health insights and personalized treatment recommendations. Also, the healthcare providers increasingly rely on decision support systems powered by artificial intelligence to analyze electronic health records (EHRs), imaging data, and diagnostic results.

Generally, AI technologies in medicine exist in many forms, from purely virtual systems for health information management to cyber-physical systems used to assist surgeons and provide active guidance in treatment decisions. The contemporary AI-based decision engines often employ machine learning and large language models (LLMs) to generate insights from structured and unstructured datasets. The large language models have shown promise in clinical contexts, with applications ranging from clinical note generation to medical text summarization, though they face significant challenges including hallucination rates of 1.47% in some implementations. In this aspect, the medical hallucinations are defined as instances where models generate misleading medical content, including incorrect dosages, drug interactions, or diagnostic criteria that can directly lead to life-threatening outcomes.

In addition, LLM-augmented clinical decision support systems have been tested in randomized controlled trials, with participants expressing concerns that “the chatbot will hallucinate, which is particularly bad in medicine,” highlighting significant trust and reliability issues. Herein, the wearable medical devices and complementary applications provide medical professionals with holistic pictures of patients' health states, enabling continuous monitoring for chronic conditions such as diabetes, cardiovascular disease, and respiratory disorders. The integration of artificial intelligence within wearable devices is revolutionizing healthcare by enabling proactive management through real-time feedback and comprehensive health monitoring.

However, current systems face significant challenges in real-time data integration. Issues related to data quality, interoperability, health equity, and fairness have been identified as major concerns, with many systems struggling to handle continuous, multi-source input streams effectively. The existing approaches are often restricted to static, limited-domain datasets and are not generalizable across various healthcare scenarios.

Furthermore, existing AI healthcare solutions demonstrate several technical and practical limitations that constrain their broader utility in clinical and consumer-facing environments. Traditional decision trees used by physicians are effectively tied to initial tree structures and are thus somewhat static, while deep learning models are less easily interpretable and may make it difficult to establish causal links. As it has been observed, AI-based clinical tools face accountability and safety challenges, with systems potentially ignoring previous states when making decisions and going against usual clinical practice by recommending sudden changes in treatment protocols. Consequently, current decision support systems often lack systematic approaches to AI implementation, resulting in niche roles rather than comprehensive integration.

In addition to the above, AI-generated outputs, particularly those involving large language models, are susceptible to generating incorrect or unsupported information, commonly referred to as “AI hallucinations,” which may lead to misinterpretations of clinical significance or inappropriate treatment recommendations. The recent studies show that large language models repeat or elaborate on planted errors in up to 83% of cases in clinical vignettes, with simple mitigation prompts halving the rate but not eliminating the risk, as published in Communications Medicine's study titled “Multi-model assurance analysis showing large language models are highly vulnerable to adversarial hallucination attacks during clinical decision support”.

The phenomenon of hallucination, where LLMs generate outputs that deviate from factual accuracy or context, poses critical challenges in high-stakes healthcare domains, requiring specialized techniques for mitigation including retrieval-augmented generation, iterative feedback loops, and supervised fine-tuning. The lack of transparent mechanisms to validate or cross-check these outputs against clinical protocols exacerbates this risk. In this aspect, many existing decision models in healthcare are built using static matrices or fixed logical pathways that cannot dynamically adapt to changes in patient data, emerging medical research, or newly introduced health metrics. The current AI approaches often trade off accuracy for transparency, making it difficult for clinicians to trust or verify inferences made by AI systems. Existing knowledge-based clinical decision support systems have shown potential to enhance practitioner performance, but many remain limited by narrow data input and non-universal applicability.

As a result, their recommendations can become outdated or misaligned with current best practices, limiting their clinical utility and reducing trust among healthcare providers. Also, the current AI systems in healthcare face limitations such as bias and lack of personalization, which must be addressed to ensure equitable and effective use. Conventional systems frequently apply generalized decision frameworks, failing to account for individual variability in physiology, lifestyle, mental health, and motivational context. Traditional personalized medicine approaches have limitations due to the complexity of analyzing vast amounts of data involved in creating personalized treatment plans.

Moreover, the risk scores or recommendations may not adjust for user-specific goals, past responses to interventions, or social support environments, thereby reducing adherence and efficacy. Integration issues between different data sources may lead to inconsistent or incomplete data, affecting the performance and reliability of AI models. Despite advances in remote patient monitoring, current systems struggle with integrating real-time data from various sources, leading to delays in generating actionable insights. Current AI systems in healthcare seldom incorporate real-time data integration at scale, with delays in data processing and limited ability to handle continuous or multi-source input streams constraining their responsiveness. This is especially problematic for chronic disease management and early intervention applications.

Additionally, most AI health engines offer little or no integration of psychosocial and behavioral health factors. This omission is significant, as adherence to health interventions is often influenced by emotional well-being, social reinforcement, and motivational state. Without incorporating these dimensions, recommendations may lack contextual sensitivity and practical relevance. While some wellness platforms offer non-invasive body monitoring or fitness planning tools, these are typically siloed from core health analytics systems and are not dynamically linked to physiological or clinical data. The absence of integration limits their utility as part of a comprehensive health management ecosystem.

There exists a clear need in the art for an intelligent health management system that overcomes the above limitations by offering robust, adaptable, and context-aware decision support. Such a system should be capable of integrating real-time physiological, psychological, behavioral, and lifestyle data across multiple modalities while incorporating structured mechanisms to reduce AI-related errors and support personalized reporting aligned with user-specific needs and goals. There is an urgent need to move away from one-shot, limited-dataset validations to dynamic scalability, longitudinal data amalgamation, and stakeholder-driven design to ensure that AI models can be ethically deployed, clinically relevant, and tractable in diverse healthcare systems.

The new systems and methods should enable dynamic updates to decision pathways, incorporate explicit safeguards against AI hallucinations, and provide mechanisms for healthcare provider oversight and validation. Additionally, there is a need to unify wellness-oriented modules, such as those supporting body contouring or fitness planning, within the broader framework of personalized health analytics. An innovation that fulfills these requirements would represent a significant advancement in the fields of preventive health, clinical decision support, and AI-based wellness optimization, addressing critical gaps in current healthcare technology while ensuring patient safety and clinical efficacy. Accordingly, there remains a need in the art for innovative, novel, inventive and efficient systems and methods for intelligent health management with real-time data integration and personalized reporting.

### SUMMARY OF INVENTION

Embodiments of the present disclosure disclose systems and methods for intelligent health management with real-time data integration and personalized reporting.

In one embodiment, a method for intelligent health management with real-time data integration and personalized reporting includes the steps of: collecting user-specific data from a plurality of sources, wherein the user-specific data comprises biometric data, clinical data, psychological data, lifestyle data, and social support data; processing the collected data using a hybrid AI engine that integrates quantitative analytical methods with large language models (LLMs), wherein the hybrid AI engine, applies natural language processing to extract contextual insights from unstructured data; employs pattern recognition algorithms to perform real-time analysis of health metrics; and, implements explicit decision guidelines to mitigate AI hallucinations; generating a reconfigurable hierarchical decision matrix that dynamically incorporates both qualitative and quantitative decision-making processes, wherein the decision matrix, maps user inputs to health risk factors using binary relevance indicators; integrates new health metrics and prognostic factors; and, evolves based on advancements in healthcare knowledge; producing personalized health reports through an output module, wherein the reports comprise actionable insights, risk assessments, and individualized recommendations tailored to user preferences and medical history; and, automatically reconfiguring decision guidelines and thresholds in real-time based on, new medical research data; user-specific health variations; and, healthcare provider inputs and feedback.

### BRIEF DESCRIPTION OF DRAWINGS

FIG. 1 illustrates flow diagram of a method for intelligent health management with real-time data integration and personalized reporting, in accordance with an embodiment of present invention.

FIG. 2 illustrates block diagram of a system for intelligent health management with real-time data integration and personalized reporting, in accordance with an embodiment of present invention.

FIG. 3 illustrates a system architecture showing the interaction between users, healthcare providers, and licensed providers with the intelligent health management system, in accordance with an embodiment of present invention.

FIG. 4 illustrates the Reconfigurable Decision Matrix structure showing a partial view of how health metrics and questionnaire items are mapped to specific health risk factors and prognostic indicators, in accordance with an embodiment of present invention.

FIG. 5 illustrates the Hierarchical Decision Approaches used to generate personalized recommendations and follow-ups by combining Analytical and Large Language Model methodologies, in accordance with an embodiment of present invention.

### DETAILED DESCRIPTION

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

While the present systems and methods have been described herein by way of example for several embodiments and illustrative drawings, those skilled in the art will recognize that the multiple embodiments disclosed hereinbelow are not limited to the embodiments or drawings described. It should be understood, that the drawings and detailed description thereto are not intended to limit embodiments to the particular form disclosed. Rather, the intention is to cover all modifications, equivalents and alternatives falling within the spirit and scope of the invention as defined by the appended claims. Any headings used herein are for organizational purposes only and are not meant to limit the scope of the description or the claims. As used herein, the word “can” and “may” is used in a permissive sense (i.e., meaning having the potential to), rather than the mandatory sense (i.e., meaning must). Similarly, the words “include”, “including”, and “includes” mean including, but not limited to.

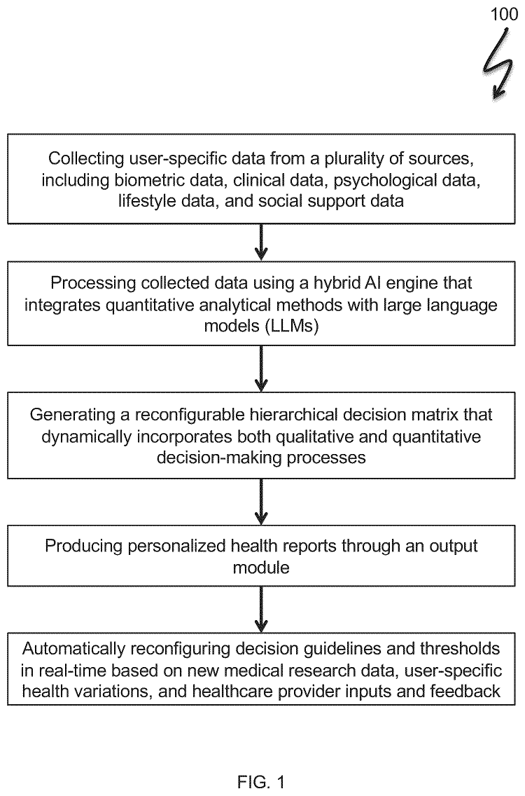

Various embodiments of the present invention relate to systems and methods for intelligent health management with real-time data integration and personalized reporting. The intelligent health management system operates through a sophisticated integration of multiple technological components designed to collect, process, analyze, and deliver personalized health insights in real-time. At the core of the invention's operation is a comprehensive data flow architecture that begins with the collection of user-specific data from a plurality of sources, as illustrated in the first step of FIG. 1. This initial phase involves gathering diverse categories of information including biometric data, clinical data, psychological data, lifestyle data, and social support data, thereby creating a holistic representation of the user's health status and contextual circumstances.

In accordance with an embodiment of the present invention, the data collection process is fundamentally enabled by a data collection module that interfaces with various input mechanisms to capture comprehensive health-related information. The module is configured to receive data from wearable devices that continuously monitor physiological parameters, manual user inputs through configurable questionnaires, electronic health record systems, diagnostic equipment outputs, and social network assessments. The configurable nature of the data collection interface allows the system to adapt to different user profiles and clinical requirements, ensuring that the gathered information is both relevant and comprehensive for subsequent processing stages.

In accordance with an embodiment of the present invention, once the user-specific data has been collected, the system advances to the processing phase depicted in the second step of FIG. 1, wherein the collected data undergoes sophisticated analysis using a hybrid AI engine that integrates quantitative analytical methods with large language models. This hybrid architecture represents a fundamental aspect of the invention's operational framework, as it combines the precision of structured analytical models with the contextual understanding capabilities of large language models. The hybrid AI engine applies natural language processing techniques to extract contextual insights from unstructured data sources such as clinical notes, patient narratives, and subjective health reports, while simultaneously employing pattern recognition algorithms to perform real-time analysis of quantifiable health metrics including vital signs, laboratory results, and biometric measurements.

In accordance with an embodiment of the present invention, the processing capabilities of the hybrid AI engine are enhanced through the implementation of explicit decision guidelines designed to mitigate AI hallucinations, which constitute a significant concern in healthcare applications of artificial intelligence. These decision guidelines function as structured constraints that align the AI engine's outputs with established medical protocols and evidence-based practices, thereby reducing the likelihood of generating incorrect or misleading information. The cross-validation mechanisms embedded within the processing step ensure that outputs from the large language models are systematically verified against the results produced by quantitative analytical methods, creating a robust error-checking framework that enhances the reliability of the system's analytical outputs.

In accordance with an embodiment of the present invention, following the data processing phase, the system proceeds to generate a reconfigurable hierarchical decision matrix as illustrated in the third step of FIG. 1, which dynamically incorporates both qualitative and quantitative decision-making processes. This decision matrix operates as an intelligent mapping system that correlates user inputs with health risk factors using binary relevance indicators, wherein each metric or response is evaluated for its relevance to specific health conditions, risk categories, or prognostic factors. The binary relevance approach enables efficient computational processing while maintaining clinical accuracy, as each data point is systematically assessed for its contribution to various health assessments.

In accordance with an embodiment of the present invention, the reconfigurable nature of the hierarchical decision matrix represents a critical innovation in the system's architecture, as it enables the integration of new health metrics and prognostic factors as they emerge from medical research and clinical practice. This adaptability is achieved through dynamic algorithms that can modify the matrix structure, adjust relevance weightings, and incorporate additional decision pathways without requiring complete system redesign or redeployment. The hierarchical organization of the decision matrix allows for multi-level analysis, wherein high-level health categories are systematically broken down into specific subcategories and individual risk factors, facilitating detailed and nuanced health assessments.

In accordance with an embodiment of the present invention, the evolution of the decision matrix based on advancements in healthcare knowledge is accomplished through automated mechanisms that monitor medical literature databases, clinical guideline updates, and research findings, subsequently integrating relevant information into the matrix structure. This continuous learning capability ensures that the system remains aligned with current medical understanding and best practices, addressing the limitation of static decision models that quickly become outdated in the rapidly evolving healthcare landscape.

In accordance with an embodiment of the present invention, upon completion of the decision matrix generation and analysis, the system advances to the fourth step illustrated in FIG. 1, wherein personalized health reports are produced through an output module. These reports comprise actionable insights that translate complex analytical results into comprehensible recommendations, risk assessments that quantify the user's likelihood of developing various health conditions, and individualized recommendations that are specifically tailored to the user's preferences, medical history, lifestyle factors, and social context. The output module employs sophisticated formatting and presentation techniques to ensure that the generated reports are accessible to both healthcare providers and end users, utilizing visualizations, plain language summaries, and hierarchically organized information structures.

In accordance with an embodiment of the present invention, the personalization of health reports extends beyond simple customization of content to encompass adaptation of communication style, level of detail, urgency indicators, and recommendation specificity based on user profiles and preferences. The output module considers factors such as health literacy levels, cultural background, motivational readiness, and personal goals when formulating the presentation and emphasis of health insights, thereby enhancing the likelihood of user engagement and adherence to recommendations.

In accordance with an embodiment of the present invention, the final operational step depicted in FIG. 1 involves the automatic reconfiguration of decision guidelines and thresholds in real-time based on three primary sources of input: new medical research data, user-specific health variations, and healthcare provider inputs and feedback. This reconfiguration mechanism operates continuously in the background, monitoring relevant data sources and triggering updates to the decision matrix when significant changes are detected. The integration of new medical research data occurs through automated literature monitoring systems that identify relevant studies, clinical trials, and guideline updates, subsequently extracting pertinent information and translating it into decision matrix modifications.

In accordance with an embodiment of the present invention, user-specific health variations are captured through the feedback loop embedded within the reconfigurable hierarchical decision matrix, which tracks user responses to recommendations, monitors changes in health metrics over time, and identifies patterns that suggest the need for adjusted thresholds or modified decision pathways. Healthcare provider inputs and feedback are integrated through the review system interface, allowing clinicians to override automated recommendations, provide contextual information not captured by standard data collection, and contribute experiential knowledge that refines the system's decision-making capabilities.

In accordance with an embodiment of the present invention, the technical solution provided by the present invention addresses fundamental limitations in current AI-driven health management systems through multiple innovative mechanisms that enhance accuracy, adaptability, and clinical utility. The hybrid AI engine architecture represents a primary technical advancement, as it mitigates the hallucination problem inherent in large language models by implementing a dual-validation approach wherein LLM outputs are systematically cross-checked against structured analytical results. This technical approach leverages the complementary strengths of both AI methodologies, utilizing large language models for their superior performance in processing unstructured data and contextual reasoning while relying on quantitative analytical methods for precise calculation of risk scores and objective health metrics.

In accordance with an embodiment of the present invention, the implementation of explicit decision guidelines within the hybrid AI engine constitutes a technical mechanism for constraining the solution space of the large language models, effectively preventing the generation of outputs that deviate from established medical knowledge and clinical protocols. These guidelines function as rule-based filters that evaluate LLM-generated content for consistency with medical literature, alignment with clinical practice standards, and logical coherence with the quantitative analysis results. The technical architecture ensures that any recommendation or insight produced by the system has undergone multiple validation stages before being presented to users or healthcare providers.

In accordance with an embodiment of the present invention, the reconfigurable hierarchical decision matrix provides a technical solution to the problem of static decision models by implementing a dynamic data structure that supports real-time modifications without requiring system downtime or manual reprogramming. The matrix employs a graph-based architecture wherein nodes represent health metrics, risk factors, or decision points, and edges represent relevance relationships or causal pathways. This structure enables efficient addition of new nodes, modification of edge weights, and restructuring of hierarchical relationships in response to new information. The binary relevance indicators utilized in the mapping process provide computational efficiency while maintaining clinical accuracy, as they enable rapid processing of large datasets through Boolean operations rather than computationally intensive probabilistic calculations.

In accordance with an embodiment of the present invention, the technical implementation of the feedback loop mechanism addresses the challenge of continuous system improvement by capturing user responses at multiple touchpoints throughout the health management process and systematically incorporating this information into future assessments. The feedback system employs machine learning algorithms that identify patterns in user responses, treatment outcomes, and adherence behaviors, subsequently adjusting decision thresholds and recommendation strategies to optimize for improved health outcomes. This adaptive learning capability ensures that the system becomes increasingly accurate and personalized over time, effectively learning from each user interaction to enhance its predictive and recommendation capabilities.

In accordance with an embodiment of the present invention, the automated reconfiguration mechanism provides a technical solution to the problem of maintaining system currency with evolving medical knowledge by implementing natural language processing pipelines that monitor medical literature databases, extract relevant information from research publications, and translate findings into decision matrix updates. The technical architecture includes validation mechanisms that assess the quality and relevance of new medical information before integration, ensuring that the system incorporates only high-quality evidence from peer-reviewed sources and recognized medical authorities.

In accordance with an embodiment of the present invention, the weighted decision criteria process represents a technical advancement in AI error mitigation by associating each decision criterion with a confidence score that reflects the reliability of the underlying data, the strength of the evidence supporting the criterion, and the consistency of the criterion with other analytical results. These confidence scores enable the system to provide users and healthcare providers with transparency regarding the certainty level of recommendations, facilitating informed decision-making and appropriate skepticism when confidence levels are low. The scoring system operates through Bayesian inference methods that combine prior probabilities based on population-level data with posterior probabilities derived from individual user data, producing nuanced confidence estimates that account for both general medical knowledge and user-specific circumstances.

In accordance with an embodiment of the present invention, the inventive step of the present invention resides in the novel combination of multiple technical features that collectively address limitations of existing AI healthcare systems in a manner that is neither obvious nor anticipated by prior art. The integration of large language models with quantitative analytical methods in a hybrid architecture represents a non-obvious solution to the hallucination problem, as conventional approaches either avoid using LLMs in clinical contexts due to reliability concerns or implement them without adequate validation mechanisms. The inventive insight underlying the hybrid approach is the recognition that LLMs and structured analytical models exhibit complementary error patterns, wherein LLMs tend to err in quantitative precision while excelling at contextual understanding, and structured models exhibit the inverse characteristic. By systematically cross-validating the outputs of these distinct AI methodologies, the invention achieves a level of reliability that exceeds what either approach could accomplish independently.

In accordance with an embodiment of the present invention, the reconfigurable hierarchical decision matrix constitutes a novel technical feature that distinguishes the present invention from static decision support systems prevalent in the prior art. While conventional systems may incorporate decision trees or lookup tables that map symptoms to diagnoses or interventions, these structures are fundamentally static and require manual reprogramming to incorporate new medical knowledge. The inventive contribution of the reconfigurable matrix lies in its dynamic architecture that enables automated integration of new health metrics, prognostic factors, and decision pathways without manual intervention. This capability is achieved through the combination of graph-based data structures, automated literature monitoring systems, and rule-based integration algorithms that work in concert to maintain system currency with medical advances.

In accordance with an embodiment of the present invention, the implementation of explicit decision guidelines to mitigate AI hallucinations represents an inventive approach that differs fundamentally from conventional error mitigation strategies such as ensemble methods or output filtering. The inventive step lies in the recognition that medical decision-making is governed by established protocols and evidence-based guidelines that can be encoded as constraints on AI system behavior. By implementing these guidelines as structural constraints within the AI engine rather than as post-hoc filters, the invention prevents the generation of invalid outputs rather than merely detecting and rejecting them after generation. This proactive approach to error mitigation is both more computationally efficient and more reliable than reactive filtering approaches.

In accordance with an embodiment of the present invention, the integration of social support data and motivational factors into the health management system represents a novel extension of traditional clinical decision support systems, which typically focus exclusively on physiological and clinical parameters. The inventive insight underlying this feature is the recognition that health outcomes are significantly influenced by psychosocial factors that are often neglected in AI-driven health systems. By systematically collecting and incorporating social support data alongside traditional health metrics, the invention enables more accurate prediction of treatment adherence and more effective personalization of recommendations to account for the user's motivational state and social context.

In accordance with an embodiment of the present invention, the weighted decision criteria process with associated confidence scores constitutes a novel approach to transparency and reliability assessment in AI healthcare systems. While some prior art systems may provide general uncertainty estimates, the invention's approach of associating individual confidence scores with each decision criterion enables granular assessment of recommendation reliability. This inventive feature allows users and healthcare providers to understand not only whether a recommendation is uncertain, but specifically which aspects of the analysis contribute to that uncertainty, facilitating more informed clinical decision-making and appropriate caution in high-stakes situations.

In accordance with an embodiment of the present invention, the best mode for implementing the intelligent health management system with real-time data integration and personalized reporting involves deploying the system as a cloud-based architecture with distributed data collection interfaces, centralized processing and analysis capabilities, and multiple user-facing applications for different stakeholder groups. In this optimal implementation, the data collection module operates through a combination of mobile applications installed on user devices, integration interfaces with wearable health monitoring devices, and secure connections to electronic health record systems maintained by healthcare providers.

In accordance with an embodiment of the present invention, the mobile data capture interface provides users with an intuitive questionnaire system that adapts in real-time based on user responses, employing branching logic to collect relevant information efficiently while minimizing user burden. The configurable questionnaires are designed to gather information across all required categories including biometric data through device sensor integration, clinical data through self-reported symptoms and conditions, psychological data through validated assessment instruments, lifestyle data through activity tracking and dietary logging, and social support data through network analysis questionnaires and relationship quality assessments.

In accordance with an embodiment of the present invention, the hybrid AI engine is optimally implemented using a combination of commercially available large language models such as GPT-4 or Claude, fine-tuned on medical literature and clinical datasets, alongside custom-developed quantitative analytical models based on established medical algorithms and risk calculators. The natural language processing capabilities are employed to analyze unstructured text inputs including user narratives, clinical notes imported from EHR systems, and responses to open-ended questionnaire items. Pattern recognition algorithms are implemented using machine learning frameworks such as TensorFlow or PyTorch, trained on large datasets of health metrics and outcomes to identify risk patterns and predict health trajectories.

In accordance with an embodiment of the present invention, the explicit decision guidelines are implemented as a comprehensive rule base derived from clinical practice guidelines published by medical professional societies, evidence summaries from organizations such as the Cochrane Collaboration, and regulatory guidance from healthcare authorities. These guidelines are encoded in a machine-readable format that enables automated validation of AI outputs against established standards, with any deviations triggering alerts for manual review or automatic rejection depending on the severity of the inconsistency.

In accordance with an embodiment of the present invention, the reconfigurable hierarchical decision matrix is optimally implemented using a graph database system such as Neo4j, which provides native support for hierarchical structures, efficient traversal algorithms, and dynamic schema modification. The binary relevance indicators are stored as edge properties in the graph, enabling rapid querying to identify all metrics relevant to a particular health risk factor or all risk factors associated with a particular metric. The integration of new health metrics and prognostic factors is accomplished through automated pipelines that monitor medical literature databases, extract relevant information using natural language processing, and generate candidate updates to the decision matrix for validation by medical experts before deployment.

In accordance with an embodiment of the present invention, the output module generates personalized health reports using template-based document generation systems that populate structured report formats with user-specific data, analytical results, and tailored recommendations. The reports are generated in multiple formats including detailed technical reports for healthcare providers, simplified summaries for end users, and interactive dashboards for ongoing monitoring. The actionable insights are formulated using natural language generation techniques that translate complex analytical results into plain language statements with appropriate context and explanatory information.

In accordance with an embodiment of the present invention, the automated reconfiguration mechanism operates through scheduled processes that execute at regular intervals to check for new medical research data, analyze user-specific health variations captured since the last update, and collect healthcare provider feedback submitted through the review system interface. The integration of new medical research involves natural language processing of research abstracts and full-text articles to identify relevant findings, automated extraction of quantitative results and clinical recommendations, and translation of these findings into decision matrix updates or threshold adjustments. User-specific health variations are analyzed using time-series analysis techniques that detect significant changes in health metrics, identify patterns suggestive of disease progression or treatment response, and trigger personalized threshold adjustments to enhance sensitivity for users at elevated risk.

In accordance with an embodiment of the present invention, the personalization module implements user preference learning through collaborative filtering techniques that identify patterns in how users respond to different types of recommendations and subsequently emphasize recommendation styles and intervention types that have historically led to positive outcomes for similar users. The review system for healthcare providers is implemented as a web-based interface that presents AI-generated reports alongside the underlying data and analytical reasoning, enabling clinicians to validate recommendations, provide corrective feedback, and approve reports for delivery to patients.

In accordance with an embodiment of the present invention, the feedback loop within the reconfigurable hierarchical decision matrix captures user responses through multiple mechanisms including adherence tracking that monitors whether users follow through with recommendations, outcome tracking that records changes in health metrics following interventions, and satisfaction surveys that assess user perceptions of recommendation relevance and utility. This feedback data is systematically analyzed to identify patterns that suggest needed adjustments to decision criteria, and successful adaptation patterns are generalized across the user population to enable system-wide improvements.

In accordance with an embodiment of the present invention, the weighted decision criteria process assigns confidence scores based on multiple factors including the sample size and quality of research evidence supporting each criterion, the consistency of the criterion with other analytical results in the current assessment, the completeness and reliability of the user data underlying the criterion, and the degree of consensus in medical literature regarding the clinical significance of the criterion. These confidence scores are presented to users through visual indicators such as color coding or star ratings, and detailed explanations of confidence levels are available through expandable information sections in the report interface.

In accordance with an embodiment of the present invention, the non-invasive body contouring recommendation module represents an optional extension of the best mode implementation, providing users interested in aesthetic wellness with evidence-based guidance on body sculpting approaches. This module collects real-time biometric data including body composition measurements, circumference measurements, and body fat distribution assessments through connected smart scales and measurement devices. The collected data is analyzed by the hybrid AI engine using specialized algorithms trained on body composition datasets and aesthetic outcome studies, generating tailored recommendations for exercise protocols, dietary modifications, and non-invasive cosmetic procedures that align with the user's aesthetic goals while maintaining compatibility with their overall health status and medical conditions.

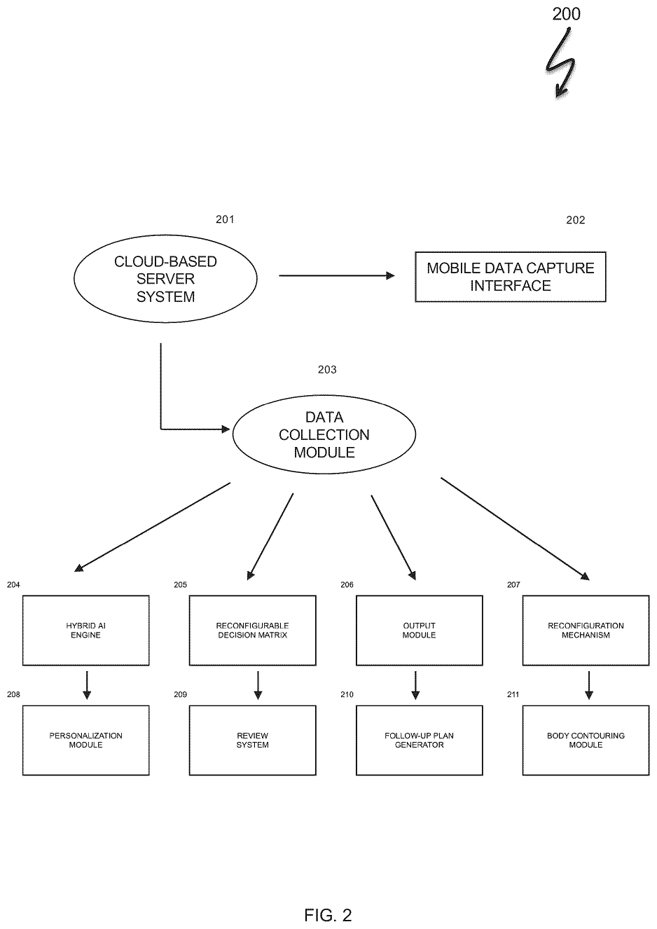

In accordance with an embodiment of the present invention, the intelligent health management system 200 with real-time data integration and personalized reporting operates through an integrated architecture comprising multiple interconnected modules that collectively enable comprehensive health assessment and management. As illustrated in FIG. 2, the system architecture is anchored by a cloud-based server system 201 that provides the computational infrastructure and data storage capabilities necessary for processing large volumes of health data and executing sophisticated AI algorithms. The cloud-based server system 201 maintains secure connections with a mobile data capture interface 202, which serves as the primary user-facing component for data input and report delivery.

In accordance with an embodiment of the present invention, the data collection module 203 functions as the central hub for gathering user-specific data from the plurality of sources, including direct inputs through the mobile data capture interface 202, integration with wearable health monitoring devices, connections to electronic health record systems, and interfaces with diagnostic equipment. The data collection module 203 is configured to aggregate biometric data such as heart rate, blood pressure, body composition measurements, and sleep patterns; clinical data including laboratory test results, imaging reports, and diagnostic findings; psychological data obtained through validated assessment instruments and mood tracking; lifestyle data encompassing dietary habits, physical activity levels, and substance use patterns; and social support data reflecting relationship quality, social network characteristics, and motivational factors.

In accordance with an embodiment of the present invention, upon successful collection of user-specific data by the data collection module 203, the system distributes this information to four primary processing components as depicted in FIG. 2. The hybrid AI engine 204 receives the collected data and performs the core analytical functions by integrating quantitative analytical methods with large language models. The hybrid AI engine 204 applies natural language processing techniques to extract contextual insights from unstructured data elements such as clinical narratives, patient-reported symptoms, and open-ended questionnaire responses, while simultaneously employing pattern recognition algorithms to analyze structured health metrics and identify risk patterns indicative of disease states or health deterioration.

In accordance with an embodiment of the present invention, the reconfigurable hierarchical decision matrix 205 operates in parallel with the hybrid AI engine 204, receiving both the raw collected data and the analytical outputs produced by the hybrid AI engine 204. The decision matrix 205 maps user inputs to health risk factors using binary relevance indicators stored within its graph-based structure, wherein each health metric or response is evaluated for its relevance to specific disease conditions, prognostic categories, or intervention recommendations. The dynamic nature of the decision matrix 205 enables integration of new health metrics and prognostic factors as they emerge from medical research, with the matrix structure automatically adapting to incorporate additional nodes, modify relationship weights, and update decision pathways without requiring manual system reconfiguration.

In accordance with an embodiment of the present invention, the output module 206 synthesizes the analytical results from both the hybrid AI engine 204 and the reconfigurable hierarchical decision matrix 205 to produce personalized health reports comprising actionable insights, quantified risk assessments, and individualized recommendations. These reports are tailored to user preferences and medical history through algorithms that consider health literacy levels, cultural factors, personal goals, and communication preferences when formulating the presentation and content emphasis of health information. The output module 206 generates reports in multiple formats suitable for different audiences, including detailed technical reports for healthcare providers and simplified summaries for end users.

In accordance with an embodiment of the present invention, the reconfiguration mechanism 207 operates continuously to automatically update decision guidelines and thresholds based on three primary information streams: new medical research data obtained through automated literature monitoring systems, user-specific health variations captured through the feedback loop within the decision matrix 205, and healthcare provider inputs received through the review system 209. The reconfiguration mechanism 207 evaluates the significance of incoming information and determines appropriate modifications to the decision matrix structure, analytical algorithms, or recommendation protocols to maintain alignment with current medical knowledge and optimize for individual user characteristics.

In accordance with an embodiment of the present invention, the personalization module 208 extends the capabilities of the output module 206 by implementing advanced customization algorithms that adjust recommendations based on individual user preferences, prior health history, treatment response patterns, and lifestyle factors. The personalization module 208 employs machine learning techniques to identify which types of interventions and communication approaches have proven most effective for users with similar characteristics, subsequently emphasizing these strategies in future recommendations.

In accordance with an embodiment of the present invention, the review system 209 provides healthcare providers with a comprehensive interface for examining AI-generated reports, validating recommendations against clinical judgment, and providing feedback that refines the system's decision-making capabilities. The review system 209 presents the complete analytical reasoning underlying each recommendation, enabling clinicians to assess the appropriateness of suggestions and identify cases requiring human expertise or clinical judgment that exceeds the system's automated capabilities.

In accordance with an embodiment of the present invention, the follow-up plan generator 210 creates structured action plans based on the personalized health reports produced by the output module 206, generating reminders, alerts, and suggested actions that guide users through the implementation of recommendations over time. The follow-up plan generator 210 adapts dynamically to changes in user health metrics by monitoring ongoing data collection and adjusting the timing, content, and urgency of follow-up communications to maintain relevance and optimize for sustained user engagement.

In accordance with an embodiment of the present invention, the body contouring module 211 represents a specialized extension of the system architecture that addresses aesthetic wellness alongside traditional health management objectives. The body contouring module 211 collects real-time biometric data specific to body composition and aesthetic parameters, analyzes this information using specialized algorithms within the hybrid AI engine 204, and generates tailored recommendations for non-invasive body sculpting approaches that align with user aesthetic goals while maintaining compatibility with overall health status.

In accordance with an embodiment of the present invention, the technical solution provided by the intelligent health management system 200 addresses critical limitations in prior art through several innovative architectural features and algorithmic approaches. The integration of the hybrid AI engine 204 with the reconfigurable hierarchical decision matrix 205 creates a dual-validation framework wherein large language model outputs undergo systematic verification against structured analytical results, effectively mitigating the hallucination problem that plagues standalone LLM implementations in healthcare contexts. This technical architecture leverages the complementary error characteristics of the two AI methodologies, utilizing LLMs for superior performance in processing ambiguous or contextual information while relying on quantitative methods for precise numerical calculations and objective risk quantification.

In accordance with an embodiment of the present invention, the implementation of explicit decision guidelines within the hybrid AI engine 204 constitutes a technical mechanism for constraining the solution space of large language models through rule-based validation systems that evaluate generated content for consistency with established medical protocols and evidence-based guidelines. This approach prevents the generation of clinically inappropriate recommendations at the source rather than merely filtering erroneous outputs after generation, providing superior computational efficiency and reliability compared to post-hoc validation approaches employed in conventional systems.

In accordance with an embodiment of the present invention, the cloud-based server system 201 provides the technical infrastructure necessary for handling computationally intensive AI operations while enabling seamless scalability to accommodate growing user populations and expanding analytical capabilities. The distributed architecture separating the mobile data capture interface 202 from the core processing components enables efficient resource utilization and maintains system responsiveness even during periods of high user activity or complex analytical operations.

In accordance with an embodiment of the present invention, the reconfigurable hierarchical decision matrix 205 employs a graph database architecture that enables efficient modification of decision structures without requiring complete system redeployment or manual reprogramming. The technical implementation supports atomic updates to individual nodes or relationships within the graph while maintaining referential integrity across the entire decision structure, ensuring that partial updates do not introduce inconsistencies or logical errors in the decision-making framework.

In accordance with an embodiment of the present invention, the feedback loop integrated within the reconfigurable hierarchical decision matrix 205 implements a continuous learning mechanism that systematically captures user responses, treatment outcomes, and adherence patterns, subsequently utilizing this information to refine decision thresholds and recommendation strategies through supervised learning algorithms. This technical approach enables the system to become progressively more accurate and personalized over time as it accumulates user interaction data and identifies patterns correlating specific user characteristics with optimal intervention strategies.

In accordance with an embodiment of the present invention, the weighted decision criteria process embedded throughout the analytical pipeline associates each decision criterion with quantitative confidence scores reflecting data quality, evidence strength, and analytical consistency. These confidence scores enable transparent communication of recommendation reliability to users and healthcare providers, facilitating appropriate trust calibration and informed decision-making regarding when to follow automated recommendations versus seeking additional clinical consultation.

In accordance with an embodiment of the present invention, the inventive contribution of the present system resides in the novel combination and interaction of multiple technical components that collectively address longstanding limitations in AI-driven healthcare systems through mechanisms not disclosed or suggested by prior art.

The hybrid architecture integrating the AI engine 204 with the reconfigurable decision matrix 205 represents a non-obvious solution to the reliability problem in medical AI, as conventional approaches either implement rigid rule-based systems lacking adaptability or deploy flexible AI models lacking adequate validation mechanisms. The inventive insight underlying this architecture is the recognition that different AI methodologies exhibit complementary strengths and weaknesses, and that systematic integration of multiple approaches can achieve reliability levels unattainable by any single methodology.

In accordance with an embodiment of the present invention, the dynamic reconfigurability of the hierarchical decision matrix 205 constitutes a novel technical feature distinguishing the present invention from static decision support systems prevalent in prior art. While conventional systems may incorporate updatable parameters or lookup tables, these structures fundamentally require manual intervention for significant structural modifications. The inventive contribution lies in the automated mechanisms enabling structural evolution of the decision matrix through integration of new medical knowledge without human programming, achieved through the combination of natural language processing pipelines monitoring medical literature, automated knowledge extraction algorithms, and graph database architectures supporting dynamic schema modification.

In accordance with an embodiment of the present invention, the integration of the mobile data capture interface 202 with the cloud-based server system 201 through secure communication protocols while maintaining support for offline data collection represents a novel approach to balancing accessibility with data security requirements. The inventive aspect resides in the synchronization mechanisms that enable seamless operation across varying network conditions while ensuring data integrity and maintaining compliance with healthcare privacy regulations.

In accordance with an embodiment of the present invention, the personalization module 208 implementing collaborative filtering techniques to identify optimal recommendation strategies based on patterns across similar users represents a novel application of recommendation system technology to clinical decision support. The inventive contribution lies in the adaptation of consumer-oriented personalization algorithms to healthcare contexts through incorporation of clinical constraints, evidence-based guidelines, and safety validation mechanisms that prevent inappropriate recommendations despite pattern-matching suggesting their potential effectiveness.

In accordance with an embodiment of the present invention, the body contouring module 211 represents a novel integration of aesthetic wellness guidance within a comprehensive health management system, addressing the technical challenge of balancing aesthetic goals with medical appropriateness through algorithms that simultaneously optimize for user satisfaction and health safety. The inventive aspect resides in the dynamic adjustment mechanisms that modify body contouring recommendations based on changes in underlying health status, ensuring that aesthetic interventions remain safe and appropriate throughout the user's health journey.

In accordance with another embodiment of the present invention, the optimal implementation of the intelligent health management system employs the cloud-based server system 201 deployed on enterprise-grade cloud infrastructure such as Amazon Web Services, Microsoft Azure, or Google Cloud Platform, utilizing containerized microservices architecture to enable independent scaling and updating of individual system components. The cloud infrastructure provides redundant storage systems, automated backup mechanisms, and distributed processing capabilities necessary for handling large user populations while maintaining high availability and system responsiveness.

In accordance with an embodiment of the present invention, the mobile data capture interface 202 is implemented as native applications for iOS and Android platforms, providing users with intuitive interfaces for data entry, questionnaire completion, and report viewing. The mobile applications maintain local data caching capabilities to enable offline operation when network connectivity is unavailable, with automatic synchronization to the cloud-based server system 201 upon restoration of network access. The interface employs adaptive questionnaire logic that modifies subsequent questions based on user responses, minimizing data collection burden while maximizing information relevance.

In accordance with an embodiment of the present invention, the data collection module 203 implements secure API integrations with wearable device manufacturers including Apple Health, Google Fit, Fitbit, and Garmin, enabling automated import of continuously collected biometric data such as heart rate, sleep patterns, physical activity levels, and body composition measurements. Electronic health record integration is achieved through HL7 FHIR standard interfaces, enabling bidirectional exchange of clinical data while maintaining compliance with healthcare interoperability requirements and privacy regulations.

In accordance with an embodiment of the present invention, the hybrid AI engine 204 is optimally implemented using large language models such as GPT-4 or Claude 3, fine-tuned on medical literature corpora and clinical datasets to enhance performance on healthcare-specific tasks. The quantitative analytical methods employ established medical algorithms including Framingham Risk Score for cardiovascular disease, FINDRISC for diabetes risk, and validated assessment instruments for mental health conditions. The natural language processing components utilize transformer-based architectures trained on clinical text datasets to extract medical concepts, identify symptom patterns, and interpret unstructured patient narratives.

In accordance with an embodiment of the present invention, the reconfigurable hierarchical decision matrix 205 employs Neo4j graph database system, providing native support for complex graph queries, efficient traversal algorithms, and dynamic schema modification without system downtime. The binary relevance indicators are implemented as edge properties with associated confidence weights, enabling probabilistic reasoning about metric relevance while maintaining computational efficiency through Boolean filtering operations.

In accordance with an embodiment of the present invention, the output module 206 generates reports using template-based document generation systems with natural language generation capabilities for producing plain language explanations of complex analytical results. Reports are rendered in HTML format for web display, PDF format for printing and archiving, and structured JSON format for integration with external systems. The visualization components employ D3.js library for creating interactive charts and graphs that enable users to explore their health data and understand analytical reasoning.

In accordance with an embodiment of the present invention, the reconfiguration mechanism 207 operates through scheduled processes executing daily to monitor medical literature databases including PubMed, Cochrane Library, and clinical guideline repositories, utilizing natural language processing to identify relevant research findings and extract actionable information for decision matrix updates. Healthcare provider feedback is collected through the review system 209 interface and systematically analyzed to identify patterns suggesting needed refinements to analytical algorithms or recommendation protocols.

In accordance with an embodiment of the present invention, the personalization module 208 implements recommendation algorithms using matrix factorization techniques that identify latent factors correlating user characteristics with intervention effectiveness, enabling prediction of optimal recommendation strategies for new users based on patterns observed in similar individuals. The follow-up plan generator 210 employs scheduling algorithms that optimize reminder timing based on circadian rhythm research, behavioral psychology principles, and individual user response patterns to maximize engagement and adherence.

In accordance with an embodiment of the present invention, the review system 209 provides clinicians with web-based dashboards presenting AI-generated reports alongside complete analytical provenance, including data inputs, algorithm selections, intermediate calculations, and confidence assessments for each recommendation. The interface enables annotation, approval, modification, and rejection of recommendations with requirement for clinical justification that feeds back into the system learning mechanisms.

In accordance with an embodiment of the present invention, the body contouring module 211 integrates with smart scales and body measurement devices supporting Bluetooth or Wi-Fi connectivity, automatically importing body composition data including body fat percentage, muscle mass, bone density, and segmental measurements. The analytical algorithms employ computer vision techniques for analyzing body proportion photographs when available, and generate recommendations encompassing exercise protocols, nutritional modifications, and non-invasive cosmetic procedures based on evidence from aesthetic medicine literature and validated outcome studies.

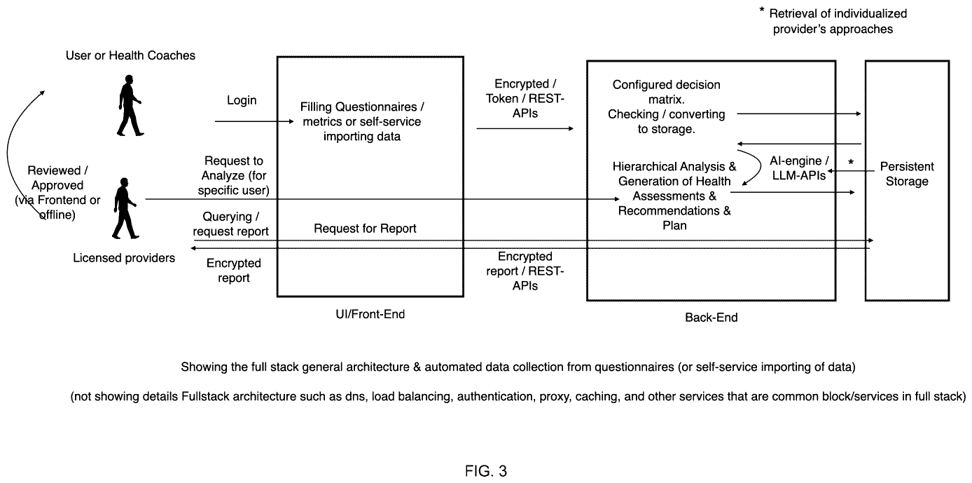

In accordance with an embodiment of the present invention, FIG. 3 illustrates the system architecture showing the interaction between users, healthcare providers, and licensed providers with the intelligent health management system. The figure depicts the data flow from user login through the UI/Front-End to the Back-End components, including the Configured Decision Matrix and Hierarchical Analysis modules that utilize AI engines and LLM/APIs for processing. The diagram shows encrypted token/REST-API communications, data storage in Persistent Storage, and the generation of encrypted reports that are delivered back to users while allowing healthcare providers to review and approve reports either through the front-end interface or offline methods.

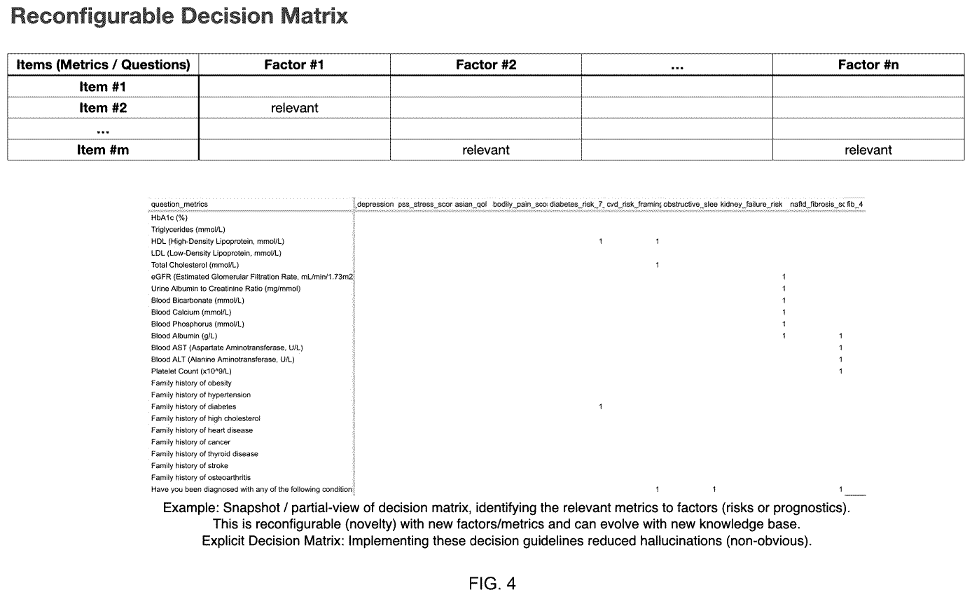

In accordance with an embodiment of the present invention, FIG. 4 illustrates the Reconfigurable Decision Matrix structure showing a partial view of how health metrics and questionnaire items are mapped to specific health risk factors and prognostic indicators. The matrix displays rows representing individual items or metrics and columns representing various factors such as depression, diabetes risk, obstructive sleep apnea, kidney failure risk, and non-alcoholic fatty liver disease. Binary relevance indicators using ‘1’ or ‘0’ values demonstrate which metrics are associated with specific health factors, enabling the system to dynamically evaluate user inputs against relevant health outcomes.

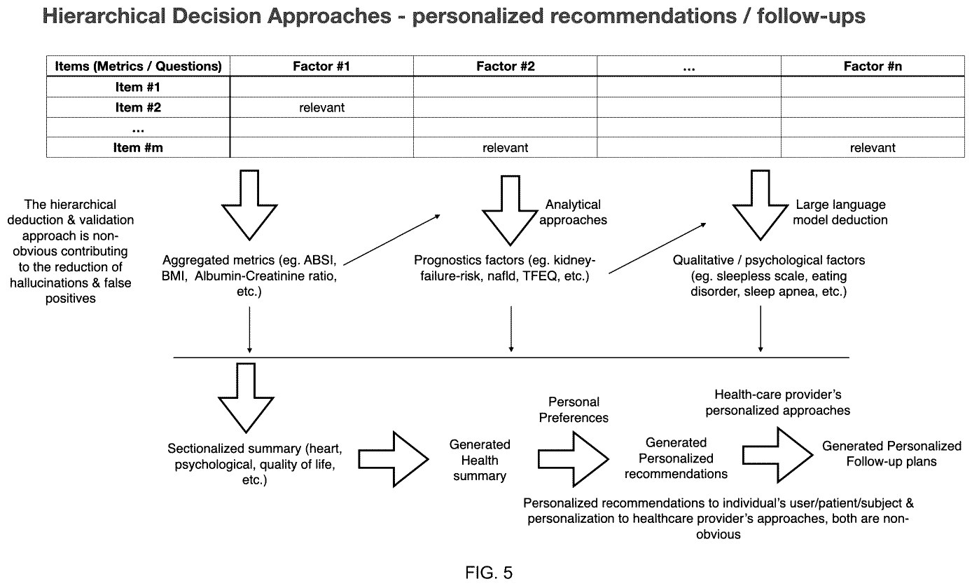

In accordance with an embodiment of the present invention, FIG. 5 illustrates the Hierarchical Decision Approaches used to generate personalized recommendations and follow-ups by combining Analytical and Large Language Model methodologies. The diagram shows the progression from the decision matrix through aggregated metrics, prognostic factors, and qualitative assessments, leading to sectionalized health summaries that incorporate psychological quality of life factors. These summaries are then transformed into personalized recommendations and ultimately into healthcare provider-specific follow-up plans, demonstrating the multi-layered analytical process that reduces AI hallucinations through cross-validation.

In accordance with an embodiment of the present invention, Table 1 presents an Auto-generated Health Assessment Report example showing the practical output of the system. The report includes a Summary of Biodata with patient demographics and health history, followed by detailed Analytical Reporting sections covering metrics summary, comorbidities risk assessments, and various disease-specific risk calculations. The table demonstrates how the system integrates computational analysis with LLM-generated insights to produce comprehensive health assessments with qualitative risk evaluations and actionable recommendations tailored to individual patient profiles.

In accordance with an embodiment of the present invention, Table 2 continues the Health Assessment Report from Table 1, displaying the Lifestyle Recommendations section that provides detailed nutritional guidance, dietary protocols, and sample meal plans. The table illustrates how the system generates practical, personalized diet planning with specific caloric requirements, macronutrient distributions, and example meals tailored to the user's health metrics and goals. This demonstrates the system's capability to translate complex analytical results into implementable action plans that users can follow for health improvement.

TABLE 1
Auto-generated Health Assessment Report (Snapshot)

| | Auto-generated Health Assessment Report (Snapshot) |
|---|---|
| Summary of | Summary of Biodata: |
| Biometric | Username: Sharon |
| | Age: 55 |
| | Sex: Female |
| | Height: 160 cm |
| | Weight: 62.5 kg |
| | History & Plan: |
| | Current Goal: Body sculpting + Weight loss |
| | Target Weight: 52 kg |
| | Target Timeline: 1 month |
| | Previous Attempts: Yes |
| | Strategies Used: Replacement meals, milkshakes, |
| | protein shakes, exercises |
| | Results: No significant result |
| | Personal Preference: |
| | Preferred Activities: None (No Exercise) |
| | Physical Limitations/Medical Conditions: None |
| | Metrics Summary: |
| | Blood Pressure: 153/91 mmHg |
| | BMI: 24.4 (Normal range: 18.5-24.9) |
| | Body Fat: 28% |
| | Visceral Fat: 12% |
| | Circumferences: |
| | Waist: 84 cm |
| | Neck: 32 cm |
| | Chest: 87.5 cm |
| | Hip: 100 cm |
| | Waist-to-Hip Ratio: 0.84 |
| | ABSI Score: 0.0789 (Z-score: −0.448, Low range: −0.868-−0.272) |
| | Fasting Blood Sugar: 6.1 mmol/L |
| | Triglycerides: 1.4 mmol/L |
| | HDL: 1.3 mmol/L |
| | Total Cholesterol: 4.1 mmol/L |
| | eGFR: >90 mL/min/1.73 m² |
| | Urine Albumin to Creatinine Ratio: 1.5 mg/mmol |
| | Blood Calcium: 2.36 mmol/L |
| | Blood Phosphorus: 1.26 mmol/L |
| | Blood Albumin: 44 g/L |
| | Blood AST: 36 U/L |
| | Blood ALT: 62 U/L |
| | Platelet Count: 206 × 10\hat{ }9/L |
| Qualitative | Analytical Reporting: |
| Risk | Comorbidities Risk: [Medium] |
| Assessments | The provided health metrics indicate a mixed risk profile for |
| integrated | comorbidities. The BMI of 24.4 falls within the normal range (18.5- |
| with LLM/ | 24.9), suggesting a healthy weight. However, the body fat percentage |
| AI-approaches | at 28% is relatively high for a female, indicating a higher amount of |
| | body fat. The visceral fat percentage at 12% is also elevated, which is |
| | concerning as visceral fat is closely linked to metabolic disorders. |
| | Additionally, the waist circumference of 84 cm is on the higher side, |
| | which can be a risk factor for cardiovascular diseases and type 2 |
| | diabetes. These factors combined suggest a medium risk for |
| | developing comorbidities. |
| | PCOS Risk: [Low] |
| | Based on the provided health questionnaire, the risk of PCOS |
| | (Polycystic Ovary Syndrome) appears to be low. The individual is a |
| | 55-year-old female with a normal BMI of 24.4. She does not report |
| | any common symptoms associated with PCOS, such as skin acne, hair |
| | loss, oily skin, difficulty conceiving, sudden weight increase, |
| | depression, difficulty sleeping, irregular menses, or excessive hair |
| | growth. Additionally, she experienced menarche at age 13 and |
| | menopause at age 55, which are within typical age ranges. Given the |
| | absence of PCOS-related symptoms and normal BMI, the risk level is |
| | assessed as low. |
| | Insulin Resistance Risk: [Medium-High] |
| | The provided health metrics indicate several risk factors associated |
| | with insulin resistance. The individual experiences forgetfulness and |
| | lack of focus, craves sugary foods, and feels stressed, which are |
| | subjective indicators of potential metabolic issues. Objectively, the |
| | individual has a blood pressure reading over 130/85 mmHg, a waist |
| | circumference exceeding 80 cm, fasting blood sugar levels above 100 |
| | mg/dL, and HDL levels below 50 mg/dL for females. These factors |
| | collectively suggest a medium-high risk for insulin resistance. |
| | However, the absence of hyperpigmentation, a diet not high in |
| | processed foods and sugar, and triglycerides below 150 mg/dL slightly |
| | mitigate the overall risk. |
| | Stress Hormone Risk: [Medium] |
| | The provided health metrics indicate a moderate risk level for stress |
| | hormone-related issues. The individual is a 55-year-old female with a |
| | BMI of 24.4, which is within the normal range. However, the body fat |
| | percentage is 28%, and visceral fat is 12%, both of which are slightly |
| | elevated. The waist circumference is 84 cm, which is on the higher |
| | side for women and can be a risk factor for stress-related health issues. |
| | The individual reports feeling stressed and experiencing unexpected |
| | upsets very often. They sometimes feel unable to control important |
| | things in their life and feel nervous and stressed. Despite these stress |
| | indicators, they often feel confident about handling personal problems |
| | and feel that things are going their way. They never feel unable to cope |
| | with tasks or control irritations, and they often feel on top of things. |
| | Overall, while there are some positive indicators of stress |
| | management, the frequent feelings of stress and inability to control |
| | important aspects of life suggest a medium risk for stress hormone- |
| | related health issues. |
| | Thyroid Risk: [Medium-Low] |
| | The individual is a 55-year-old female with a BMI of 24.4, which falls |
| | within the normal range. She has a body fat percentage of 28% and a |
| | visceral fat percentage of 12%. Her waist circumference is 84 cm, and |
| | she does not exhibit symptoms commonly associated with thyroid |
| | issues, such as hair loss, dry skin, or a double chin. There is no family |
| | history of thyroid disease, and she does not report significant fatigue |
| | or constipation. However, her blood pressure is elevated at 153/91 |
| | mmHg, which could be a concern. Overall, the risk of thyroid disease |
| | appears to be medium-low based on the provided data. |
| | Sex Hormone Risk: [Medium-Low] |
| | Based on the provided health metrics and questionnaire responses, the |
| | sex hormone risk appears to be medium-low. The individual has a |
| | normal BMI of 24.4 and a body fat percentage of 28%, which is within |
| | a reasonable range for females. The visceral fat percentage is 12%, and |
| | the waist circumference is 84 cm, both of which are slightly elevated |
| | but not excessively high. The individual does not report common |
| | symptoms associated with hormonal imbalances such as skin acne, |
| | hair loss, oily skin, difficulty conceiving, sudden weight increase, |
| | depression, difficulty sleeping, irregular menses, or hirsutism. |
| | However, the individual does report feeling stressed and experiencing |
| | decreased libido, which can be influenced by hormonal fluctuations. |
| | Overall, while there are some signs of stress and decreased libido, the |
| | absence of more severe symptoms and the relatively normal body |
| | composition metrics suggest a medium-low risk for sex hormone- |
| | related issues. |
| | Leptin Risk: [Medium-High] |
| | The provided health metrics indicate several factors that contribute to |
| | a medium-high leptin risk. The individual reports feeling stressed and |
| | experiencing body pain and headaches, which are associated with |
| | higher leptin levels. Additionally, the craving for high-calorie or oily |
| | food and alcohol consumption can further influence leptin resistance. |
| | Despite having a normal BMI and body fat percentage, the elevated |
| | blood pressure (153/91 mmHg) and fasting blood sugar (6.1 mmol/L) |
| | suggest metabolic disturbances that can affect leptin regulation. The |
| | absence of digestive issues and bloating is positive, but the overall |
| | profile points to a significant risk of leptin imbalance. |
| | Metaflammation (Fatty Liver) Risk: [Medium-High] |
| | The provided health metrics indicate a medium-high risk for |
| | metaflammation and fatty liver. The subject is a 55-year-old female |
| | with a BMI of 24.4, which is within the normal range. However, the |
| | body fat percentage is 28%, and visceral fat is 12%, both of which are |
| | relatively high. The waist circumference is 84 cm, exceeding the |
| | recommended limit for females, which is a significant risk factor. |
| | Additionally, the subject has high blood pressure (153/91 mmHg) and |
| | fasting blood sugar levels over 100 mg/dL, both of which are |
| | indicative of metabolic syndrome. HDL levels are also below the |
| | recommended threshold for females. The subject reports cravings for |
| | sugary and high-calorie foods, experiences stress, and consumes |
| | alcohol occasionally. Physical activity levels are low, with minimal |
| | engagement in moderate or vigorous activities. These factors |
| | collectively contribute to an elevated risk of developing fatty liver and |
| | associated metabolic conditions. |
| | Epworth Sleepiness Scale (ESS) score: [5 out of 24: Lower] |
| | The Epworth Sleepiness Scale (ESS) score of 5 indicates a lower level |
| | of daytime sleepiness. This score is derived from the individual's |
| | responses, which mostly indicate a slight chance of dozing in various |
| | situations, with no instances of dozing while lying down to rest, sitting |
| | and talking to someone, or in a car stopped in traffic. This suggests |
| | that the individual experiences minimal daytime sleepiness. |
| | Physical Activity Metrics [MET-minutes/week]: Vigorous [0.0], |
| | Moderate [60.0], Walking [0.0], Total [60.0] |
| | In the past week, there were no vigorous physical activities reported. |
| | Moderate physical activities were performed on one day for less than |
| | 30 minutes, resulting in 60 MET-minutes. No walking activities were |
| | recorded. The total physical activity for the week amounts to 60 MET- |
| | minutes, indicating minimal engagement in physical activities. |
| | Dietary Habits: [Need-Improvement] |
| | Based on the provided data, the dietary habits show a mix of both |
| | positive and negative aspects. The individual does not consume a high |
| | amount of processed foods or sugary foods daily, which is good. |
| | However, there are several areas that need improvement. The craving |
| | for high-calorie or oily foods and the occasional consumption of |
| | alcohol could be contributing to less optimal health metrics. The |
| | individual also consumes soda or sugary drinks daily and does not |
| | drink more than 2 liters of water daily, which are areas of concern. |
| | Additionally, the reliance on restaurant food and the occasional |
| | consumption of sugary foods and processed foods indicate a need for |
| | more balanced and home-cooked meals. |
| | Recommendations: |
| | 1. Increase Water Intake: Aim to drink at least 2 liters of water |
| | daily. |
| | 2. Reduce Sugary Drinks: Limit the consumption of soda and |
| | sugary drinks. |
| | 3. Home-Cooked Meals: Increase the frequency of home-cooked |
| | meals to ensure better control over ingredients and portion |
| | sizes. |
| | 4. Balanced Diet: Incorporate more fruits, whole grains, and a |
| | variety of vegetables into daily meals. |
| | 5. Limit High-Calorie Foods: Reduce the intake of high-calorie |
| | and oily foods to improve overall health metrics. |
| | Eating Disorder Score (with TFEQ-R18): Cognitive Restraint: |
| | [19/24], Uncontrolled Eating: [12/36], Emotional Eating: [3/12] |
| | The individual's eating disorder score, based on the TFEQ-R18 |
| | questionnaire, indicates a high level of cognitive restraint (19/24), |
| | suggesting a strong tendency to control food intake to manage weight. |
| | The score for uncontrolled eating (12/36) is moderate, implying |
| | occasional difficulties in regulating food consumption. The emotional |
| | eating score (3/12) is low, indicating that emotions have minimal |
| | impact on their eating behavior. Overall, the data suggests a |
| | disciplined approach to eating with occasional lapses in control, but |
| | minimal emotional influence on eating habits. |
| Aggregated | Diabetes Risk in 7.5 Years: [49.26%] |
| computational | Based on the provided health metrics, the risk of developing diabetes |
| & LLM / AI- | in the next 7.5 years is 49.26%. Factors contributing to this risk |
| approaches | include age (55 years), elevated blood pressure (153/91 mmHg), and |
| (or Analytical + | fasting blood sugar level (6.1 mmol/L). Despite a normal BMI (24.4) |
| Qualitative) | and no family history of diabetes, the combination of these factors |
| for Risks & | significantly increases the risk. |
| Prognosis | Framingham Risk Score for CVD in 10 years: [7.22%] |
| factors (from | The Framingham Risk Score estimates a 7.22% chance of developing |
| hierarchical | cardiovascular disease (CVD) within the next 10 years for a 55-year- |
| decision | old female with the provided health metrics. Key factors include |
| approaches | elevated blood pressure (153/91 mmHg), HDL cholesterol of 1.3 |
| described | mmol/L, and total cholesterol of 4.1 mmol/L. The absence of smoking |
| prior) | and other diagnosed conditions positively influences the risk score. |
| | Obstructive Sleep Apnea: [High] |
| | The provided health metrics indicate a high risk of obstructive sleep |
| | apnea (OSA). Key factors contributing to this assessment include: |
| | Age: 55 years, which is a higher risk category for OSA. |
| | Sex: Female, though males are generally at higher risk, post- |
| | menopausal women have increased risk. |
| | BMI: 24.4, within the normal range, but close to the upper |
| | limit. |
| | Neck Circumference: 32 cm, which is not particularly high |
| | but should be considered in conjunction with other symptoms. |
| | Symptoms: |
| | Snoring: Yes, a common symptom of OSA. |
| | Feeling constantly tired: Yes, another significant |
| | indicator of potential OSA. |
| | Observed Apneas: No, but absence of observed apneas |
| | does not rule out OSA. |
| | Given the presence of snoring and constant tiredness, it is advisable to |
| | follow up with a sleep study to confirm the diagnosis and determine |
| | the appropriate treatment. |
| | Kidney Failure Risk Score in 5 years: [<1%] |
| | Based on the provided health metrics, the kidney failure risk score |
| | within five years is calculated to be less than 1%. The key factors |
| | contributing to this low risk include an estimated glomerular filtration |
| | rate (eGFR) of greater than 90 mL/min/1.73 m², which indicates |
| | normal kidney function, and a urine albumin to creatinine ratio of 1.5 |
| | mg/mmol, which is within the normal range. Additionally, other |
| | relevant blood parameters such as blood calcium (2.36 mmol/L), blood |
| | phosphorus (1.26 mmol/L), and blood albumin (44 g/L) are within |
| | normal limits, further supporting a low risk of kidney failure. |
| | Non-alcoholic fatty liver disease (NAFLD) fibrosis score: [-2.35, |
| | Low risk] |
| | The NAFLD fibrosis score is calculated using age, BMI, blood |
| | glucose levels, platelet count, albumin, AST, and ALT levels. Given |
| | the data: - Age: 55 - BMI: 24.4 - Fasting Blood Sugar: 6.1 mmol/L - |
| | Platelet Count: 206 × 10\hat{ }9/L - Blood Albumin: 44 g/L - Blood AST: 36 |
| | U/L - Blood ALT: 62 U/L |
| | The calculated NAFLD fibrosis score is −2.35, indicating a low risk of |
| | advanced fibrosis. |
| | FIB-4 score: [1.221], APRI score: [0.437] |
| | The FIB-4 score of 1.221 and APRI score of 0.437 are calculated using |
| | the provided data: age, AST, ALT, and platelet count. These scores are |
| | used to assess liver fibrosis in patients with Non-alcoholic fatty liver |
| | disease (NAFLD). The FIB-4 score is derived from age, AST, ALT, |
| | and platelet count, while the APRI score is calculated using AST and |
| | platelet count. Both scores help in evaluating the extent of liver |
| | fibrosis. |
| | Environmental & Social Support: [Very Good] |
| | The individual demonstrates a strong environmental and social support |
| | system for dietary control. They do not smoke and consume alcohol |
| | infrequently, which minimizes potential negative influences on their |
| | health goals. Additionally, they have close friends or family members |
| | to discuss weight goals and progress with, as well as to exercise with |
| | and help keep them accountable. This social network can provide |
| | motivation and practical support. The individual is also highly |
| | motivated to change their diet and lifestyle, driven by health concerns |
| | and the desire for improved self-esteem. This combination of social |
| | support and personal motivation significantly enhances their capacity |
| | to achieve and maintain dietary control. |
| | CES-D Depression Score: [3 out of 39: No Risk] |
| | The individual's responses to the CES-D scale indicate minimal |
| | depressive symptoms. Most responses fall under “None or less than 1 |
| | day,” suggesting a low frequency of depressive feelings. Positive |
| | indicators such as feeling good about oneself, hopeful about the future, |
| | and happy for most of the week further support the low depression |
| | score. The total score of 3 out of a possible 39 points places the |
| | individual in the “No Risk” category for depression. |
| | Perceived Stress Scale (PSS): [14 out of 40: Moderate Stress] |
| | Based on the provided responses, the individual's Perceived Stress |
| | Scale (PSS) score is calculated to be 14 out of 40, indicating a |
| | moderate level of stress. The responses show variability in stress |
| | experiences, with frequent feelings of being upset by unexpected |
| | events and occasional feelings of nervousness and stress. However, the |
| | individual often feels confident in handling personal problems and |
| | perceives things as going their way, suggesting a balanced stress |
| | perception overall. |
| | Physical Functioning (QOL) Score: [20 out of 21: High |
| | Functioning] |
| | The individual demonstrates high physical functioning capabilities, |
| | with minimal limitations. They report no restrictions in walking long |
| | distances, climbing stairs, lifting groceries, or engaging in vigorous |
| | and moderate activities. The only noted limitation is a slight restriction |
| | in bending, kneeling, or stooping. This minor limitation results in a |
| | near-perfect score, indicating overall excellent physical health and |
| | mobility. |
| | Bodily Pain (QOL) Score: [1 out of 6: Low Pain Level] |
| | The individual reported experiencing moderate body pain over the past |
| | four weeks. However, this pain did not interfere with their normal |
| | work, including housework, nor did it affect their social activities. This |
| | indicates that while the pain is present, it has a minimal impact on |
| | their overall quality of life and daily functioning. |
| AI-Generated | Analytical Summary: |
| Health | Risks & Further Investigations: [Blood Pressure, Insulin |
| Assessment | Resistance, Leptin, Metaflammation, Obstructive Sleep Apnea, |
| Summary | Stress Hormones] |
| (with | Sharon's health profile indicates several areas of medium to high risk |
| aggregated | that require further investigation and follow-up. Her blood pressure is |
| factors | elevated at 153/91 mmHg, which is above the normal range and |
| described | suggests a risk for hypertension. This necessitates regular monitoring |
| prior) | and possibly lifestyle or pharmacological interventions. Additionally, |
| | her fasting blood sugar level of 6.1 mmol/L and a 49.26% risk of |
| | developing diabetes in 7.5 years indicate a medium-high risk for |
| | insulin resistance, warranting further glucose tolerance tests and |
| | dietary adjustments. |
| | Sharon's elevated ALT levels (62 U/L) and a medium-high risk for |
| | metaflammation (fatty liver) suggest potential liver health issues, |
| | which should be monitored through liver function tests and possibly |
| | imaging studies. Her high risk for obstructive sleep apnea, indicated |
| | by snoring and constant tiredness, requires a sleep study to confirm the |
| | diagnosis and determine appropriate treatment. The medium risk for |
| | stress hormones and leptin resistance, combined with her moderate |
| | stress levels (PSS score of 14), suggests that stress management |
| | strategies and possibly hormonal evaluations are necessary to support |
| | her weight loss and overall health goals. |
| | Weight Type Classification: [Normal Weight, Metabolically |
| | Unhealthy Obesity (MUO)] |
| | Based on Sharon's BMI of 24.4, she falls within the normal weight |
| | range (18.5-24.9). However, her health metrics indicate |
| | characteristics of Metabolically Unhealthy Obesity (MUO). Despite |
| | having a normal BMI, Sharon's elevated blood pressure (153/91 |
| | mmHg), fasting blood sugar (6.1 mmol/L), and HDL levels (1.3 |
| | mmol/L) suggest metabolic health issues. Additionally, her waist |
| | circumference of 84 cm exceeds the healthy threshold for females (80 |
| | cm), indicating central fat accumulation, which is a risk factor for |
| | metabolic syndrome. |
| | MUis characterized by the presence of metabolic abnormalities such |
| | as hypertension, dyslipidemia, insulin resistance, and elevated fasting |
| | glucose levels, even in individuals with a normal BMI. Sharon's |
| | elevated blood pressure and fasting blood sugar levels, combined with |
| | her central obesity (waist circumference), place her at increased risk |
| | for cardiovascular diseases and type 2 diabetes. Addressing these |
| | metabolic issues through lifestyle changes, such as improved diet and |
| | increased physical activity, is crucial for reducing her health risks. |
| | Personal Weight Plan: [30% Success] |
| | Sharon's goal is to achieve body sculpting and weight loss, targeting a |
| | weight of 52 kg within a month. Given her current weight of 62.5 kg, |
| | this represents a significant reduction of 10.5 kg in a very short period. |
| | Sharon has previously attempted weight loss using replacement meals, |
| | protein shakes, and exercises but did not see significant results. She |
| | also does not enjoy physical activities, which could be a challenge in |
| | achieving her goal. |
| | The optimistic success chance is estimated at 30%. This estimate |
| | considers Sharon's high motivation and willingness to make |
| | significant lifestyle changes, as well as her good environmental and |
| | social support. However, the ambitious timeline, combined with her |
| | lack of enjoyment in physical activities and previous unsuccessful |
| | attempts, makes this goal particularly challenging. Additionally, her |
| | current health metrics, such as high blood pressure and moderate stress |
| | levels, may also impact her ability to achieve rapid weight loss. A |
| | more gradual and sustainable approach, incorporating dietary |
| | adjustments and moderate physical activity, might increase the |
| | likelihood of long-term success. |

TABLE 2
Auto-generated Health Assessment Report (cont.)

| | Auto-generated Health Assessment Report (cont.) | | | | |
|---|---|---|---|---|---|
| AI-Generated | LifeStyle Recommendations: | | | | |
| Personalized | Daily Caloric Needs for Sharon | | | | |
| health | 0 Caloric Needs: 1,800 kcal/day | | | | |
| recommendati | 0 Caloric Needs (for weight loss): 1,300 kcal/day | | | | |
| ons (example | Macronutrient Recommendations | | | | |
| above for diet, | Macronutrient | Amount (g) | | | |
| can be | Carbohydrate | 130 g | | | |
| extended to | Total Fiber | 25 g | | | |
| fitness and | Protein | 100 g | | | |
| others | Fat | 50 g | | | |
| depending on | Water | 2 L | | | |
| providers. | These recommendations are designed to improve insulin sensitivity | | | | |
| | and promote weight loss while meeting Sharon's caloric needs. | | | | |
| | JomSlim Protocol for Sharon | | | | |
| | 1. Protocol Ratio: | | | | |
| | Protein: 30% | | | | |
| | Vegetables: 50-60% | | | | |
| | Carbohydrates: 10-20% | | | | |
| | 2. Dietary Changes to Improve Insulin Sensitivity: | | | | |
| | Carbohydrates: Low (>55% from non-starchy vegetables) | | | | |
| | Fiber: High | | | | |
| | Protein: Moderate | | | | |
| | Fat: Healthy sources | | | | |
| | 3. Recommended Foods: | | | | |
| | Emphasize: Bitter foods, dark green vegetables, celery, garlic, | | | | |
| | olive oil, black coffee, green tea. | | | | |
| | Meal Sequence: Start with vegetables, followed by meat, and | | | | |
| | end with carbohydrates. | | | | |
| | 4. Supplement Protocol: | | | | |
| | Probiotic | | | | |
| | Fish Oil | | | | |
| | Vitamins: D, B, B6, C, E | | | | |
| | Calcium Citrate | | | | |
| | Magnesium | | | | |
| | Zinc | | | | |
| | Chromium | | | | |
| | 5. Lifestyle Adjustments: | | | | |
| | Minimize Plastic Usage and Chemical Exposure: Reduce | | | | |
| | environmental hormones (e.g., in shampoo, dish soap). | | | | |
| | Sleep: Ensure sleep by 10 PM, aiming for at least 8 hours of | | | | |
| | quality rest. | | | | |
| | Meal Timing Options: | | | | |
| | First meal after 12 PM, avoid eating after 8 PM; or | | | | |
| | First meal after 7:30 AM, avoid eating after 5 PM. | | | | |
| | 6. Food Choices: | | | | |
| | Avoid: Juices, sugary and salty foods, fast food, processed | | | | |
| | foods, underground vegetables. | | | | |
| | Refer further details and recommendations in the attached sections, | | | | |
| | and to licensed physicians & doctors for details. | | | | |
| AI-Generated | JomSlim 30 Days Program by MediSpring | | | | |
| Protocol, | Self-Managed Protocols | | | | |
| Planning, | These protocols can be self-managed with consultation from Lifestyle | | | | |
| follow-ups | & Health Coaches at JomSlim. | | | | |
| from specific | 6. RPG Customized Exercises at Home: | | | | |
| healthcare | Engage in a 10-minute personalized exercise routine | | | | |
| provider | designed by a sports medicine specialist and a board- | | | | |
| tailored | certified personal trainer. This routine is tailored to | | | | |
| | your fitness level, goals, and specific health | | | | |
| | considerations. | | | | |
| | The exercises are optimized for efficiency and | | | | |
| | effectiveness, targeting key muscle groups, improving | | | | |
| | flexibility, and enhancing cardiovascular health. With | | | | |
| | an emphasis on proper form and technique, this | | | | |
| | bespoke workout ensures maximum benefits within a | | | | |
| | short timeframe, making it ideal for busy schedules or | | | | |
| | those new to exercise. | | | | |
| | 7. Food Points for Personalized Food Intake: | | | | |
| | Benefit from comprehensive dietary guidance provided | | | | |
| | by a team of nutritionists, dietitians, physicians, and | | | | |
| | weight management specialists. These professionals | | | | |
| | offer personalized dietary recommendations aimed at | | | | |
| | promoting optimal health, managing weight, and | | | | |
| | addressing specific health concerns. | | | | |
| | By incorporating evidence-based nutrition strategies | | | | |
| | and considering individual preferences and lifestyle | | | | |
| | factors, this collaborative approach to nutrition | | | | |
| | empowers you to make informed food choices and | | | | |
| | cultivate sustainable, long-term dietary habits for | | | | |
| | overall well-being. | | | | |
| | 8. Non-Invasive Machines for Reducing Fat and Building | | | | |
| | Muscles: | | | | |
| | Utilize non-invasive machines, such as JomSlim | | | | |
| | HiEMTech innovated by Japan and the USA, to reduce | | | | |
| | fat and build muscle, thereby sculpting your body | | | | |
| | shape. | | | | |
| | These treatments provide effective body contouring | | | | |
| | without the need for surgery, minimizing downtime and | | | | |
| | risks associated with invasive procedures. These | | | | |
| | devices offer a practical, safe, and effective solution for | | | | |
| | enhancing body contours and muscle definition. | | | | |
| | Consultation with Licensed Medical Professionals | | | | |
| | These protocols should be consulted and administered by licensed | | | | |
| | medical professionals. Seek consultation with MediSpring directly. | | | | |
| | 9. Medical Grade Oral Medicine for Weight Management: | | | | |
| | These medications are essential for treating obesity and | | | | |
| | related conditions, often approved by health authorities | | | | |
| | like the FDA. They work through various mechanisms | | | | |
| | such as appetite suppression, fat absorption inhibition, | | | | |
| | or metabolic enhancement. | | | | |
| | Typically recommended by clinical guidelines for | | | | |
| | individuals who have not achieved adequate weight | | | | |
| | loss through lifestyle changes alone, they provide an | | | | |
| | essential option for effective and sustainable weight | | | | |
| | management under medical supervision. | | | | |
| | 10. Physician Injectable Medicine for Weight Management: | | | | |
| | Physician-administered injectable medications are a | | | | |
| | critical component in the treatment of obesity, often | | | | |
| | regarded as a gold standard due to their effectiveness. | | | | |
| | Highly recommended in medical guidelines for patients | | | | |
| | who have not had success with oral medications or | | | | |
| | lifestyle interventions, these injectables offer a potent | | | | |
| | and targeted approach to weight loss, helping to | | | | |
| | achieve significant and clinically meaningful results. | | | | |
| | 11. Gastric Balloon for Obesity by Surgeon: | | | | |
| | This non-surgical intervention involves placing a | | | | |
| | balloon in the stomach to reduce its capacity, | | | | |
| | promoting a feeling of fullness and aiding in weight | | | | |
| | loss. | | | | |
| | Minimally invasive and recommended for individuals | | | | |
| | who have not succeeded with diet and exercise alone, it | | | | |
| | is widely endorsed in clinical guidelines due to its | | | | |
| | relative safety and effectiveness in achieving moderate | | | | |
| | weight loss. | | | | |
| | 12. Gastrectomy Operation for Severe Obesity by Sub- | | | | |
| | Specialty GI Surgeon: | | | | |
| | This procedure, considered a gold standard by many | | | | |
| | medical guidelines, involves removing a portion of the | | | | |
| | stomach to significantly reduce its size and capacity, | | | | |
| | leading to substantial and sustained weight loss. | | | | |
| | Highly recommended for individuals with severe | | | | |
| | obesity who have not responded to other treatments, it | | | | |
| | not only aids in weight loss but also improves or | | | | |
| | resolves related comorbidities, such as type 2 diabetes | | | | |
| | and hypertension. | | | | |
| | 13. Personalized Oral Supplement for Wellness: | | | | |
| | These supplements support overall health and fill | | | | |
| | nutritional gaps that may not be met through diet alone. | | | | |
| | Tailored to contain essential vitamins, minerals, and | | | | |
| | other nutrients, they promote various aspects of well- | | | | |
| | being, including immune function, energy levels, and | | | | |
| | cognitive health. | | | | |
| | Highly recommended by health guidelines as an | | | | |
| | adjunct to maintain optimal health and prevent | | | | |
| | deficiencies. | | | | |
| | 14. Personalized Oral Detox Package for Wellness: | | | | |
| | This personalized approach supports the body's natural | | | | |
| | detoxification processes, improving metabolic health, | | | | |
| | reducing inflammation, and promoting cellular repair | | | | |
| | and regeneration. | | | | |
| | Often recommended for individuals seeking the | | | | |
| | benefits of fasting without complete food abstinence, it | | | | |
| | is a practical and effective tool for enhancing overall | | | | |
| | health and well-being. | | | | |
| | 15. Intravenous Supplements for Wellness: | | | | |
| | This treatment involves delivering vitamins, minerals, | | | | |
| | and other nutrients directly into the bloodstream, | | | | |
| | bypassing the digestive system for immediate | | | | |
| | absorption and maximum effectiveness. | | | | |
| | Recommended by healthcare professionals for | | | | |
| | individuals with low energy levels, weakened immune | | | | |
| | systems, or specific nutrient deficiencies, IV vitamin | | | | |
| | therapy can provide quick relief and support for various | | | | |
| | health concerns. | | | | |
| | 16. Stem Cell Treatment - New Emerging Alternative: | | | | |
| | Stem cell therapy is a groundbreaking approach that | | | | |
| | holds promise for treating a wide range of diseases and | | | | |
| | conditions by harnessing the regenerative potential of | | | | |
| | stem cells. | | | | |
| | While still considered an emerging field, it has shown | | | | |
| | encouraging results in clinical trials for conditions such | | | | |
| | as osteoarthritis, autoimmune diseases, and | | | | |
| | neurological disorders. Further research is needed to | | | | |
| | fully understand its safety and efficacy. | | | | |
| | 17. Head to Toe Full Body Screening and Cancer Screening: | | | | |
| | Comprehensive health evaluation and early disease | | | | |
| | detection, including blood tests, urine tests, endoscopy, | | | | |
| | sleep studies, ECG, scans, and imaging. | | | | |
| | Highly recommended by medical guidelines for | | | | |
| | individuals at risk or seeking proactive health | | | | |
| | management, these screenings are valuable tools in | | | | |
| | preventive healthcare. | | | | |
| | 18. ENT Endoscopy: | | | | |
| | This diagnostic and therapeutic procedure examines the | | | | |
| | ear, nose, and throat (ENT) regions, aiding in the | | | | |
| | accurate diagnosis and treatment of various conditions | | | | |
| | such as rhinitis, sinusitis, tumors, cancer, ear infections, | | | | |
| | and vocal cord disorders. | | | | |
| | Highly recommended for its minimally invasive nature | | | | |
| | and ability to provide real-time insights, making it an | | | | |
| | essential tool in otolaryngology. | | | | |
| | 19. Upper and Lower Gastro-Intestinal Endoscopy: | | | | |
| | Upper GI endoscopy focuses on the esophagus, | | | | |
| | stomach, and duodenum, while lower GI endoscopy | | | | |
| | (colonoscopy) examines the colon and rectum. | | | | |
| | Considered the gold standard for diagnosing issues like | | | | |
| | ulcers, tumors, polyps, and inflammatory bowel | | | | |
| | disease, GI endoscopy provides direct visualization and | | | | |
| | biopsy capabilities, making it indispensable for | | | | |
| | accurate diagnosis and effective management of | | | | |
| | gastrointestinal disorders. | | | | |
| AI-Generated | Sample Meals: | | | | |
| Personalized | Meal 1: Breakfast | | | | |
| diet planning | Dish: Kaya Toast with Soft-Boiled Eggs and Black Coffee | | | | |
| (can be | Ingredients: | | | | |
| extended to | 2 slices of wholemeal bread | | | | |
| fitness | 1 tbsp kaya (coconut jam) | | | | |
| planning and | 2 soft-boiled eggs | | | | |
| others, | 1 cup black coffee (no sugar) | | | | |
| depending on | Calories: 350 kcal | | | | |
| providers) | Macronutrients: Protein: 15 g | | | | |
| | Carbohydrates: 40 g | | | | |
| | Fat: 15 g | | | | |
| | Meal 2: Lunch | | | | |
| | Dish: Chicken Rice (Healthier Version) | | | | |
| | Ingredients: | | | | |
| | 1 cup brown rice | | | | |
| | 100 g steamed chicken breast | | | | |
| | 1 cup steamed broccoli | | | | |
| | 1 tbsp light soy sauce | | | | |
| | 1 tsp sesame oil | | | | |
| | Calories: 450 kcal | | | | |
| | Macronutrients: | | | | |
| | Protein: 30 g | | | | |
| | Carbohydrates: 50 g | | | | |
| | Fat: 10 g | | | | |
| | Meal 3: Dinner | | | | |
| | Dish: Stir-Fried Vegetables with Tofu and Brown Rice | | | | |
| | Ingredients: | | | | |
| | 1 cup brown rice | | | | |
| | 150 g firm tofu | | | | |
| | 1 cup mixed vegetables (carrots, bell peppers, snow peas) | | | | |
| | 1 tbsp olive oil | | | | |
| | 1 tbsp soy sauce | | | | |
| | Calories: 500 kcal | | | | |
| | Macronutrients: | | | | |
| | Protein: 20 g | | | | |
| | Carbohydrates: 60 g | | | | |
| | Fat: 20 g | | | | |
| | Summary Table: | | | | |
| | Meal | Calories (kcal) | Protein (g) | Carbohydrates (g) | Fat (g) |
| | Breakfast | 350 | 15 | 40 | 15 |
| | Lunch | 450 | 30 | 50 | 10 |
| | Dinner | 500 | 20 | 60 | 20 |
| | Total | 1300 | 65 | 150 | 45 |

Notes:
The total daily intake of 1300 kcal is designed to support
weight loss while providing balanced nutrition.
The meals include a mix of protein, carbohydrates, and fats to
ensure satiety and energy throughout the day.
Incorporating whole grains, lean proteins, and vegetables helps
in maintaining a healthy diet.
Adjustments can be made based on individual preferences and
dietary needs.

Utility: The app provides a useful function in automating health assessments,
organizing and generating accurate healthcare assessment reports quickly in
minutes (vs. hours to days by trained healthcare providers)
Enablement: The detailed description in the disclosure provides sufficient
information for someone skilled in the art to implement this invention.

Those of ordinary skills in the art will appreciate that the present invention provides substantial improvements over existing AI-driven health management systems through its hybrid architecture that integrates large language models with quantitative analytical methods. This dual-validation approach significantly reduces the occurrence of AI hallucinations that plague conventional systems relying solely on large language models, thereby enhancing the reliability and clinical safety of automated health recommendations. The cross-validation mechanism ensures that insights generated through natural language processing undergo systematic verification against structured analytical results, creating a robust error-checking framework that maintains accuracy levels necessary for high-stakes healthcare applications.

In use, the reconfigurable hierarchical decision matrix represents a significant advancement in system adaptability, enabling dynamic incorporation of new medical research and evolving clinical guidelines without requiring manual reprogramming or system downtime. This capability addresses a critical limitation of static decision support systems that quickly become outdated as medical knowledge advances. The graph-based architecture supporting atomic updates to individual decision nodes while maintaining referential integrity across the entire structure ensures that the system remains aligned with current best practices and emerging evidence, providing users with recommendations that reflect the latest medical understanding.

In further use, the comprehensive data integration capabilities of the invention enable holistic health assessment by simultaneously considering biometric, clinical, psychological, lifestyle, and social support factors. This multi-dimensional approach surpasses conventional systems that focus narrowly on physiological parameters, as it recognizes that health outcomes are influenced by complex interactions among physical, mental, and social determinants. The inclusion of social support data and motivational factors enables more accurate prediction of treatment adherence and more effective personalization of recommendations, addressing the behavioral aspects of health management that traditional systems neglect.

Also, the real-time processing capabilities facilitated by the cloud-based architecture ensure timely delivery of health insights and recommendations, which is particularly critical for chronic disease management and early intervention scenarios. The system's ability to continuously monitor incoming data streams from wearable devices and other sources, automatically triggering analysis and generating alerts when significant changes are detected, enables proactive health management rather than reactive responses to established health problems. This responsiveness provides substantial advantages over systems with delayed data processing that may miss critical windows for intervention.

The personalization mechanisms embedded throughout the system architecture deliver recommendations tailored to individual user characteristics, preferences, and circumstances rather than applying generalized protocols. The collaborative filtering techniques identifying optimal intervention strategies based on patterns across similar users enable the system to predict which approaches are most likely to succeed for each individual, thereby improving adherence rates and health outcomes. This level of personalization surpasses conventional systems that apply population-level guidelines without adequate consideration of individual variability in physiology, psychology, and social context.

The transparency provided through weighted decision criteria and associated confidence scores enables users and healthcare providers to make informed judgments about when to follow automated recommendations versus seeking additional clinical consultation. This transparency mechanism addresses trust issues that often hinder adoption of AI healthcare systems, as it provides clear visibility into the reliability of specific recommendations rather than presenting all outputs with uniform confidence. The scoring system quantifying reliability based on data quality, evidence strength, and analytical consistency facilitates appropriate calibration of trust in system outputs.

The healthcare provider review system integration ensures that automated recommendations undergo expert validation before implementation in clinical practice, maintaining the essential role of human judgment while leveraging AI capabilities for efficiency and consistency. The interface enabling clinician annotation, modification, and approval of AI-generated reports with feedback mechanisms that refine future system performance creates a collaborative framework wherein AI augments rather than replaces clinical expertise. This human-in-the-loop approach addresses concerns about overreliance on automated systems while still capturing efficiency benefits.

The automated follow-up plan generation capability maintains user engagement over extended timeframes through strategic timing of reminders, alerts, and suggested actions that adapt dynamically to changing health metrics. This sustained engagement addresses a critical challenge in health management wherein initial motivation often wanes over time, leading to poor adherence and suboptimal outcomes. The adaptive scheduling algorithms optimizing reminder timing based on behavioral psychology principles and individual response patterns maximize the likelihood of sustained participation in health improvement efforts.

The scalability enabled by the cloud-based infrastructure allows the system to serve large user populations while maintaining responsiveness and enabling continuous expansion of analytical capabilities. The microservices architecture supporting independent scaling and updating of individual components provides operational flexibility that reduces maintenance complexity and enables rapid deployment of improvements. This scalability advantage over monolithic systems facilitates broader adoption and ensures that performance remains consistent as user populations grow.

The cost efficiency achieved through automation of routine health assessment and reporting functions reduces the burden on healthcare providers, enabling them to focus their expertise on complex cases requiring nuanced clinical judgment. The system's capability to generate comprehensive health reports within minutes, compared to the substantial time investment traditionally required for manual analysis and documentation, represents significant resource savings for healthcare organizations. This efficiency improvement enables broader access to personalized health management services that would be economically infeasible with traditional labor-intensive approaches.

While the system and methods described herein are preferably implemented in software, implementations in hardware or a combination of software and hardware are also possible and contemplated. All examples and conditional language recited herein are intended for pedagogical objects to aid the reader in understanding the example embodiments and the concepts contributed by the inventor to furthering the art, and are to be construed as being without limitation to such specifically recited examples and conditions.

Accordingly, while there has been shown and described the preferred embodiment of the invention is to be appreciated that the invention may be embodied otherwise than is herein specifically shown and described and, within said embodiment, certain changes may be made in the form and arrangement of the parts without departing from the underlying ideas or principles of this invention within the scope of the claims appended herewith.

What is claimed is:

1. A method for intelligent health management with real-time data integration and personalized reporting, said method comprising the steps of:
collecting user-specific data from a plurality of sources, wherein said user-specific data comprises biometric data, clinical data, psychological data, lifestyle data, and social support data;
processing said collected data using a hybrid AI engine that integrates quantitative analytical methods with large language models (LLMs), wherein said hybrid AI engine, applies natural language processing to extract contextual insights from unstructured data; employs pattern recognition algorithms to perform real-time analysis of health metrics; and, implements explicit decision guidelines to mitigate AI hallucinations;
generating a reconfigurable hierarchical decision matrix that dynamically incorporates both qualitative and quantitative decision-making processes, wherein said decision matrix, maps user inputs to health risk factors using binary relevance indicators; integrates new health metrics and prognostic factors; and, evolves based on advancements in healthcare knowledge;
producing personalized health reports through an output module, wherein said reports comprise actionable insights, risk assessments, and individualized recommendations tailored to user preferences and medical history; and,
automatically reconfiguring decision guidelines and thresholds in real-time based on, new medical research data; user-specific health variations; and, healthcare provider inputs and feedback.

2. The method of claim 1, wherein said plurality of parameters further comprises one or more of laboratory test results, blood test results, urine test results, stool test results, imaging reports, scan reports, endoscopy results, DNA testing results, genetic testing results, sleep study data, electrophysiological testing data, histopathology reports, and cytology reports.

3. The method of claim 1, wherein said processing step further comprises: cross-validating outputs from said large language models with said quantitative analytical methods to reduce AI overgeneralization; aggregating multiple health risk factors and prognostic metrics into a structured summary; and modifying decision pathways based on user-specific preferences and healthcare provider inputs to customize recommendations for individual physiological and psychological profiles.

4. The method of claim 1, further comprising: adjusting said individualized recommendations using a personalization module based on individual user preferences, prior health history, and lifestyle factors; and providing a review system for healthcare providers to review and approve AI-generated reports, wherein said review system includes an interface for provider feedback to enable additional adjustments and ensure compliance with medical standards.

5. The method of claim 1, wherein said reconfigurable hierarchical decision matrix includes a feedback loop that captures user responses and incorporates new data points into future assessments for continuous improvement and adaptation of decision criteria based on real-time health insights and advancements in medical research.

6. The method of claim 1, further comprising: generating an automated follow-up plan that provides users with reminders, alerts, and suggested actions based on said personalized health reports; and adapting said follow-up plan to changes in user health metrics to modify suggestions accordingly.

7. The method of claim 1, further comprising: incorporating a weighted decision criteria process to mitigate AI hallucinations at each stage of analysis, wherein each criterion is associated with a confidence score for enhancing accuracy and reliability of AI-generated health assessments; and quantifying reliability and confidence levels of AI-generated insights, wherein said reconfigurable hierarchical decision matrix includes a scoring system based on real-time physiological data providing users with metrics representing accuracy of medical recommendations.

8. The method of claim 1, further comprising: collecting real-time biometric data and user preferences related to body sculpting using a non-invasive body contouring recommendation module; analyzing said collected biometric data using said hybrid AI engine to generate tailored body contouring recommendations; and dynamically adjusting said body contouring recommendations based on updated user data and progress measurements.

9. An intelligent health management system with real-time data integration and personalized reporting, said system comprising:
a data collection module configured to gather user-specific data from a plurality of sources, wherein said user-specific data comprises biometric data, clinical data, psychological data, lifestyle data, and social support data;
a hybrid AI engine that integrates quantitative analytical methods with large language models (LLMs), wherein said hybrid AI engine is configured to apply natural language processing to extract contextual insights from unstructured data, employ pattern recognition algorithms to perform real-time analysis of health metrics, and implement explicit decision guidelines to mitigate AI hallucinations;
a reconfigurable hierarchical decision matrix that dynamically incorporates both qualitative and quantitative decision-making processes, wherein said decision matrix is configured to map user inputs to health risk factors using binary relevance indicators, integrate new health metrics and prognostic factors, and evolve based on advancements in healthcare knowledge;
an output module configured to produce personalized health reports, wherein said reports comprise actionable insights, risk assessments, and individualized recommendations tailored to user preferences and medical history; and
a reconfiguration mechanism configured to automatically reconfigure decision guidelines and thresholds in real-time based on new medical research data, user-specific health variations, and healthcare provider inputs and feedback.

10. The system of claim 9, wherein said plurality of parameters further comprises one or more of laboratory test results, blood test results, urine test results, stool test results, imaging reports, scan reports, endoscopy results, DNA testing results, genetic testing results, sleep study data, electrophysiological testing data, histopathology reports, and cytology reports.

11. The system of claim 9, wherein said hybrid AI engine is further configured to cross-validate outputs from said large language models with said quantitative analytical methods to reduce AI overgeneralization, aggregate multiple health risk factors and prognostic metrics into a structured summary, and modify decision pathways based on user-specific preferences and healthcare provider inputs to customize recommendations for individual physiological and psychological profiles.

12. The system of claim 9, further comprising a personalization module configured to adjust said individualized recommendations based on individual user preferences, prior health history, and lifestyle factors, and a review system for healthcare providers configured to review and approve AI-generated reports, wherein said review system includes an interface for provider feedback to enable additional adjustments and ensure compliance with medical standards.

13. The system of claim 9, wherein said reconfigurable hierarchical decision matrix includes a feedback loop configured to capture user responses and incorporate new data points into future assessments for continuous improvement and adaptation of decision criteria based on real-time health insights and advancements in medical research.

14. The system of claim 9, further comprising an automated follow-up plan generator configured to provide users with reminders, alerts, and suggested actions based on said personalized health reports, and to adapt said follow-up plan to changes in user health metrics to modify suggestions accordingly.

15. The system of claim 9, further comprising a weighted decision criteria process configured to mitigate AI hallucinations at each stage of analysis, wherein each criterion is associated with a confidence score for enhancing accuracy and reliability of AI-generated health assessments, and wherein said reconfigurable hierarchical decision matrix includes a scoring system based on real-time physiological data providing users with metrics representing accuracy of medical recommendations.

16. The system of claim 9, further comprising a non-invasive body contouring recommendation module configured to collect real-time biometric data and user preferences related to body sculpting, analyze said collected biometric data using said hybrid AI engine to generate tailored body contouring recommendations, and dynamically adjust said body contouring recommendations based on updated user data and progress measurements.
